(19) United States
(12) Patent Application Publication
(10) Pub. No.: US 2019/0307320 A1
(43) Pub. Date: Oct. 10, 2019

(54) IMAGE PICKUP APPARATUS, ENDOSCOPE AND ENDOSCOPE SYSTEM

(71) Applicant: OLYMPUS CORPORATION, Tokyo (JP)

(72) Inventor: Satoru ADACHI, Tsuchiura-shi (JP)

(73) Assignee: OLYMPUS CORPORATION, Tokyo (JP)

(21) Appl. No.: 16/449,928

(22) Filed: Jun. 24, 2019

Related U.S. Application Data
(63) Continuation of application No. PCT/JP2017/035160, filed on Sep. 28, 2017.

(30) Foreign Application Priority Data
Dec. 28, 2016 (JP) 2016-256916

(51) Int. Cl. A61B 1/045; G02B 23/24; A61B 1/06; A61B 1/00; H04N 5/335; H04N 9/07

(52) U.S. Cl. CPC A61B 1/045; G02B 23/2461; A61B 1/0676; H04N 2005/2255; H04N 5/335; H04N 9/07; A61B 1/00006; A61B 1/00186

(57) ABSTRACT

An image pickup apparatus includes: an image pickup device in which a high-sensitivity pixel group and a normal sensitivity pixel group are arranged; a control section configured to read the high-sensitivity pixel group alone to form a first frame signal for which a configuration of the first pixel group to be read according to a control signal synchronized with a light source selection control signal for selecting a type of illuminating light from a light source capable of emitting a plurality of types of illuminating light for illuminating an object, read all pixels including the high-sensitivity pixel group and the normal sensitivity pixel group to form a second frame signal and alternately output the first and second frame signals; and a frame addition circuit configured to perform frame addition processing of the first and second frame signals to output an image signal for one frame.

### CROSS REFERENCE TO RELATED APPLICATION

This application is a continuation application of PCT/JP2017/035160 filed on Sep. 28, 2017 and claims benefit of Japanese Application No. 2016-256916 filed in Japan on Dec. 28, 2016, the entire contents of which are incorporated herein by this reference.

### BACKGROUND OF THE INVENTION

### 1. Field of the Invention

The present invention relates to an image pickup apparatus, an endoscope and an endoscope system and specifically relates to an image pickup apparatus including a solid-state image pickup device provided with an on-chip color filter, an endoscope and an endoscope system.

### 2. Description of the Related Art

Endoscope systems each including, e.g., an endoscope configured to pick up an image of an object inside a subject and an image processing apparatus configured to generate an observation image of the object picked up by the endoscope have widely been used in a medical field, an industrial field, etc.

Also, for an endoscope in the type of endoscope system, conventionally, an example in which as a solid-state image pickup device configured to receive light of an object image and output a predetermined image signal, for example, a CMOS image sensor (complementary metal-oxide semiconductor image sensor) is employed has been known.

Also, as CMOS image sensors such as above, in recent years, as indicated in Japanese Patent Application Laid-Open Publication No. 2009-176777, image sensors provided with what is called on-chip color filters have widely been known.

The on-chip color filters are color selection filters formed on respective sensors of the pixels in an image pickup device (for example, a CMOS image sensor as mentioned above). Normally, for example, sensor sections in an image pickup device such a CMOS image sensor output only information of black and white tones of received light; however, disposition of on-chip colors filter on the sensor sections enables providing “color information” to respective pixels in the sensor sections via the on-chip color filters.

Also, generally, an example in which on-chip color filters are configured by primary color filters of respective R, G and B colors have widely been known; however, in recent years, a technique that makes a part of on-chip color filters to be a transparent layer (forming of what is called white pixels) to enhance sensitivity as an image pickup device has been proposed. Furthermore, a technique that employs what is called complementary color filters of, e.g., cyan, magenta and yellow as on-chip color filters for sensitivity enhancement has also been proposed.

The specification of Japanese Patent No. 5526673 indicates a technique in which two types of pixels having different sensitivities (one type of which is a white pixel) are arranged in advance and if the pixel having a higher sensitivity is saturated, a signal of the pixel having a lower sensitivity is used.

The specifications of Japanese Patent Nos. 5256917 and 4618342 each indicate an example in which pixel design itself is changed to enhance a saturated number of charges for a photoelectric conversion section used for a pixel having a higher sensitivity so that saturation is suppressed.

### SUMMARY OF THE INVENTION

An image pickup apparatus according to an aspect of the present invention includes: an image pickup device including a color filter array of color filters, in which a first pixel group and a second pixel group including respective sensitivities that are different from each other are arranged according to characteristics of the color filters; a reading timing control circuit configured to control a first reading timing for a first frame formed by reading the first pixel group alone, the first frame allowing a configuration of the first pixel group to be read to be appropriately changed according to a control signal synchronized with a light source selection control signal for selecting a type of illuminating light from a light source capable of emitting a plurality of types of illuminating light for illuminating an object, and a second reading timing for a second frame formed by reading all pixels including the first pixel group and the second pixel group; an output control circuit configured to, based on the control by the reading timing control circuit, perform control to alternately output a first frame signal for the first frame read at the first reading timing and a second frame signal for the second frame read at the second reading timing; and a frame addition circuit configured to perform frame addition processing of the first frame signal and the second frame signal controlled and outputted by the output control circuit to output an image signal for one frame.

An endoscope according to an aspect of the present invention includes the image pickup apparatus.

An endoscope system according to an aspect of the present invention includes the endoscope.

### BRIEF DESCRIPTION OF THE DRAWINGS

FIG. 1 is an outer appearance perspective view illustrating a configuration of an endoscope system including an image pickup apparatus according to a first embodiment of the present invention;

FIG. 2 is a block diagram illustrating an electric configuration of an endoscope system including the image pickup apparatus according to the first embodiment;

FIG. 3 is an electric circuit diagram illustrating an electric configuration of an image pickup device in the image pickup apparatus according to the first embodiment;

FIG. 4 is a diagram illustrating transition of the respective numbers of optical charges in high-sensitivity pixels and normal pixels in the image pickup device, the numbers of optical charges being increased with time, and decimated reading timings and all-pixel reading timings, in the image pickup apparatus according to the first embodiment;

FIG. 5 is a timing chart illustrating an example of respective control signals applied to the image pickup device in all-pixel reading, in the image pickup apparatus according to the first embodiment;

FIG. 6 is a timing chart illustrating an example of respective control signals applied to the image pickup device in decimated reading, in the image pickup apparatus according to the first embodiment;

FIG. 7 is a diagram indicating transmittances of respective colors that are primary colors, blue and green, in addition to cyan and magenta where an image pickup device employs complementary color filters in an image pickup apparatus according to a second embodiment of the present invention;

FIG. 8 is a diagram illustrating decimated reading timings and all-pixel reading timings when a light source selects white light where the image pickup device employs complementary color filters in the image pickup apparatus according to the second embodiment;

FIG. 9 is a diagram illustrating pixel reading timings when the light source selects NBI light where the image pickup device employs complementary color filters in the image pickup apparatus according to the second embodiment;

FIG. 10 is a block diagram illustrating an electric configuration of an endoscope system including an image pickup apparatus according to a modification;

FIG. 11 is a block diagram illustrating an electric configuration of an endoscope system including another image pickup apparatus according to a modification;

FIG. 12 is a block diagram illustrating an electric configuration of an endoscope system including another image pickup apparatus according to a modification; and

FIG. 13 is a block diagram illustrating an electric configuration of an endoscope system including another image pickup apparatus according to a modification.

### DETAILED DESCRIPTION OF THE PREFERRED EMBODIMENTS

Embodiments of the present invention will be described below with reference to the drawings.

### First Embodiment

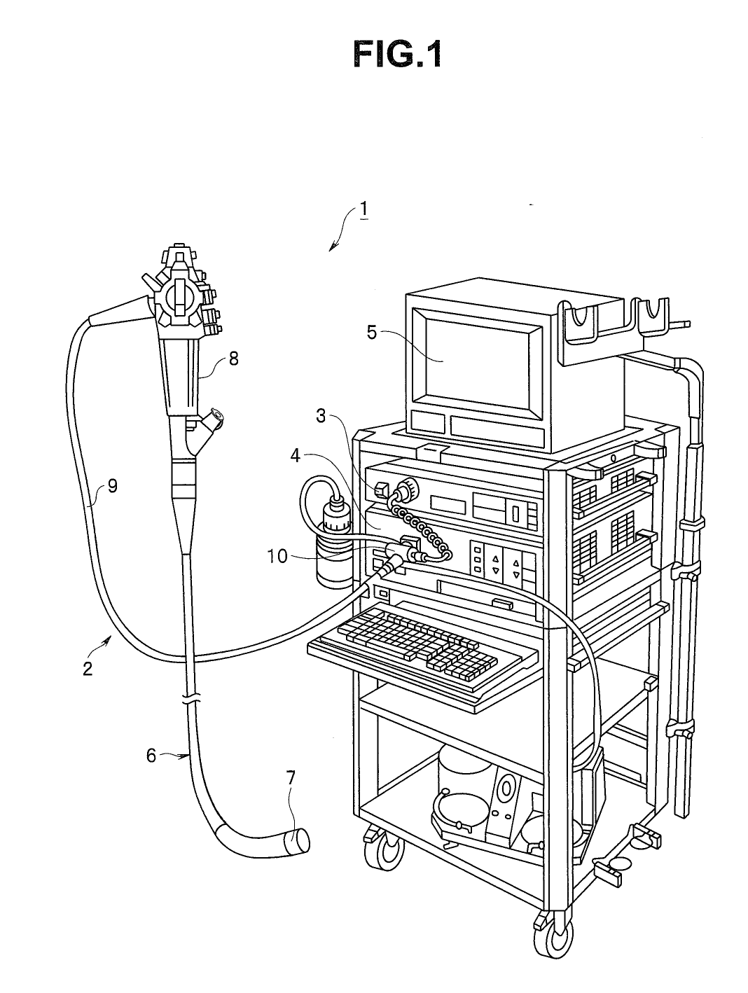

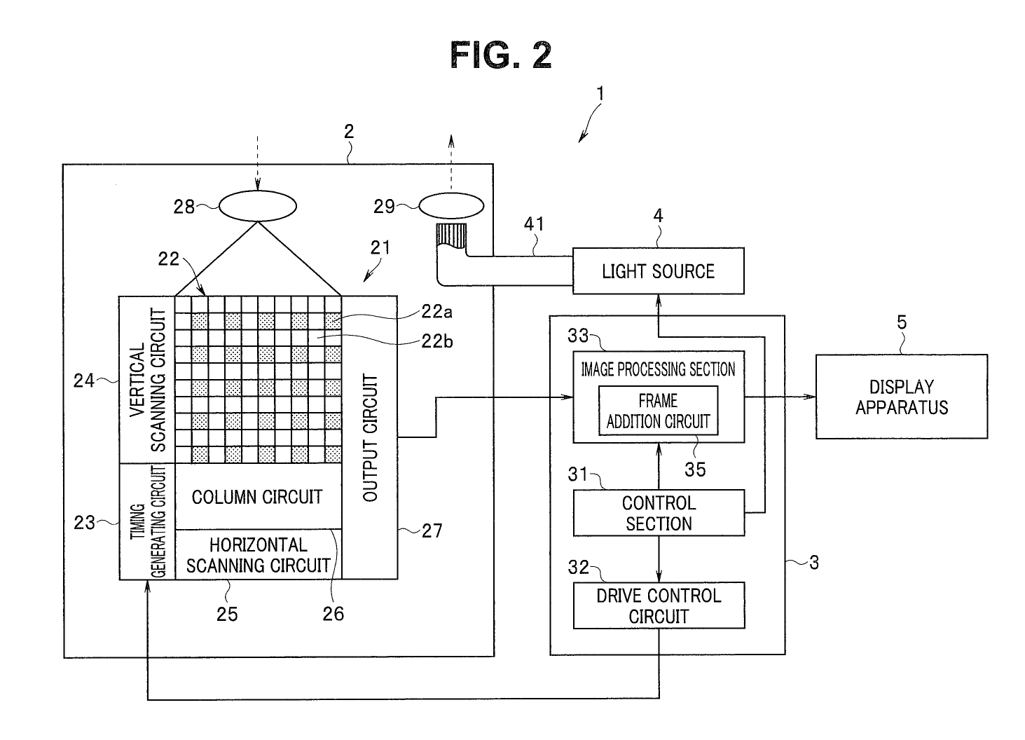

FIG. 1 is an outer appearance perspective view illustrating a configuration of an endoscope system including an image pickup apparatus according to a first embodiment of the present invention, and FIG. 2 is a block diagram illustrating an electric configuration of an endoscope system including the image pickup apparatus according to the first embodiment.

Note that the present embodiment will be described taking an endoscope including a solid-state image pickup device as an image pickup apparatus, the endoscope being configured to pick up an image of an object inside a subject, and an endoscope system as an example.

As illustrated in FIGS. 1 and 2, an endoscope system 1 including the image pickup apparatus (endoscope) according to the first embodiment includes: an endoscope 2 configured to allow observation of a subject and pick up an image of the subject; a video processor 3 connected to the endoscope 2, the video processor 3 being configured to receive an input of a signal of the image pickup and subject to the image pickup signal to predetermined image processing; a light source apparatus 4 configured to supply illuminating light for illuminating the subject; and a display apparatus 5 configured to display an observation image according to the image pickup signal.

The endoscope 2 includes an elongated insertion portion 6 to be inserted into, e.g., a body cavity of the subject, a rigid distal end portion 7 provided on the distal end side of the insertion portion 6, an endoscope operation portion 8 disposed on the proximal end side of the insertion portion 6, the endoscope operation portion 8 being grasped and operated by a surgeon, and a universal cord 9, one end of which is provided so as to extend from a side portion of the endoscope operation portion 8.

On the proximal end side of the universal cord 9, a connector 10 is provided, and the connector 10 is connected to the light source apparatus 4. In other words, a fitting (not illustrated) that is an end portion of connection with a fluid conduit projecting from a distal end of the connector 10 and a light guide fitting (not illustrated) that is an end portion of supply of illuminating light are detachably connected to the light source apparatus 4.

Furthermore, an end of a connection cable is connected to an electric contact portion provided at a side face of the connector 10. Inside the connection cable, for example, a signal wire configured to transmit an image pickup signal from an image pickup device 21 (see FIG. 2) in the endoscope 2 is provided, and a connector portion at the other end of the connection cable is connected to the video processor 3.

Referring back to FIG. 2, the endoscope 2 includes an illuminating optical system 29 disposed at a distal end portion of a light guide 41 provided so as to extend from the light source apparatus 4, an objective optical system 28 including a lens configured to receive light of an object image, and an image pickup device 21 disposed at an image plane of the objective optical system 28, which are disposed in the distal end portion 7 of the insertion portion 6.

In the present embodiment, the image pickup device 21 is a solid-state image pickup device including a CMOS image sensor. Also, the image pickup device 21 includes what is called an on-chip color filter array, and a high-sensitivity pixel group (first pixel group) and a normal sensitivity pixel group (second pixel group) having respective sensitivities that are different from each other depending on characteristics of the color filters are arranged.

As illustrated in FIG. 2, the image pickup device 21 includes, e.g., a light receiving section 22, a timing generating circuit 23, a vertical scanning circuit 24, a horizontal scanning circuit 25, a column circuit 26 and an output circuit 27; however, specific configurations of the components will be described later.

The video processor 3 includes a control section 31 configured to control various circuits inside the video processor 3, a drive control circuit 32 configured to generate a drive signal for controlling the image pickup device 21 in the endoscope 2 under the control of the control section 31, an image processing section 33 configured to receive an input of an image pickup signal generated and outputted by the image pickup device 21 and subjects the image pickup signal to predetermined image processing under the control of the control section 31, and a frame addition circuit 35 provided in the image processing section 33 and configured to add up a plurality of frame signals. Note that the frame addition circuit 35 will be described in detail later.

In the present embodiment, the light source apparatus 4 includes a white light source configured to generate white light as illuminating light to be applied to an object, and a narrow-band observation light source configured to generate narrow-band light to be provided for what is called NBI (narrow-band imaging: narrow-band light observation), the narrow-band light being configured by blue light and green light, as illuminating light to be applied to an object.

Also, the illuminating light generated by each of the light sources in the light source apparatus 4 is applied as predetermined illuminating light (the white light or the narrow-band light) via the light guide 41, by the endoscope 2.

### <Specific Configuration of Image Pickup Device 21>

Next, a configuration of the image pickup device 21 in the present embodiment will be described.

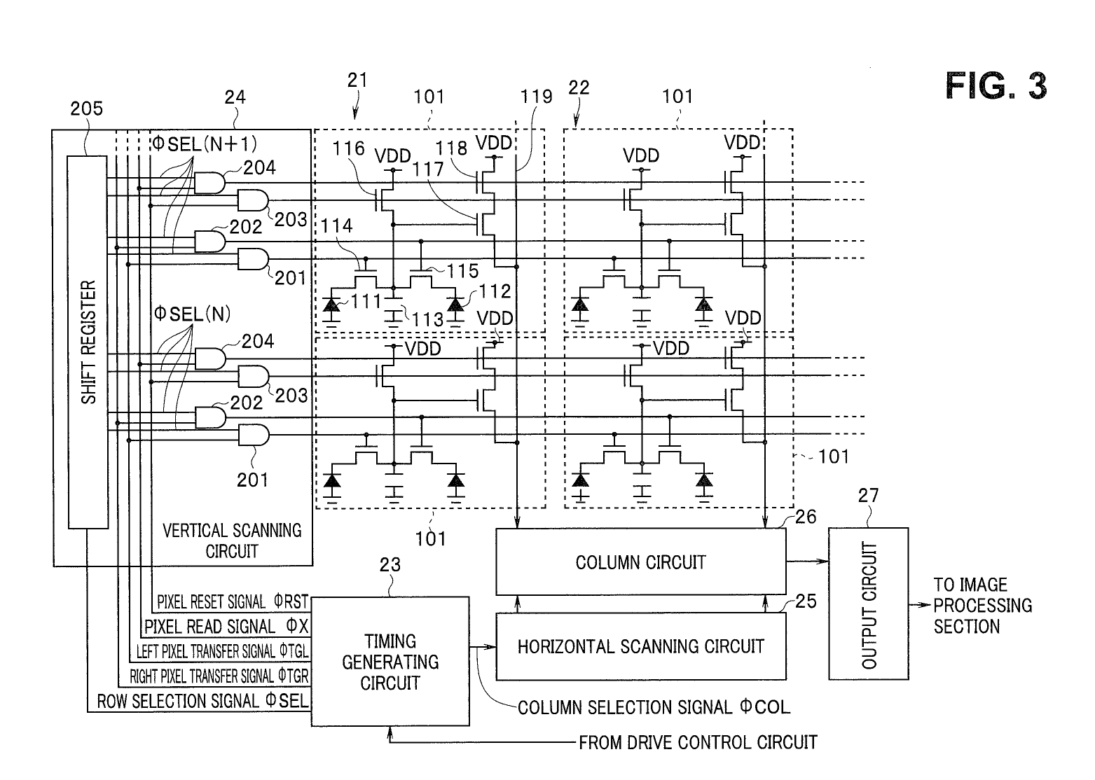

FIG. 3 is an electric circuit diagram illustrating an electric configuration of the image pickup device in the image pickup apparatus according to the first embodiment. The image pickup device 21 in the present embodiment will be described below with reference to FIG. 2 in addition to FIG. 3.

As described above, in the present embodiment, the image pickup device 21 is a solid-state image pickup device including a CMOS image sensor (complementary metal-oxide semiconductor image sensor). Also, in the present embodiment, the image pickup device 21 employs what is called a 4-transistor CMOS image sensor as a base, and in the present embodiment, an example in which horizontal 2-pixel sharing pixels are arranged is employed.

As illustrated in FIGS. 2 and 3, the image pickup device 21 includes the light receiving section 22. The light receiving section 22 includes a plurality of unit pixel (unit cell) 101 arrayed in a two-dimensional matrix. Here, in the present embodiment, each unit pixel 101 includes a plurality of photoelectric conversion elements (photo diodes (PD)) and charge transfer transistors corresponding to the photoelectric conversion elements.

More specifically, as described above, in the present embodiment, the horizontal 2-pixel sharing pixels are arranged, and each unit pixel 101 includes two, left and right, photoelectric conversion elements (photo diodes), and left and right charge transfer transistors corresponding to the left and right photo diodes, respectively.

In other words, a plurality of unit pixels 101, each of which is a unit pixel 101 that is a unit pixel in the present embodiment, each include two, left and right, “pixels” capable of outputting “pixel output components” that are different from each other.

In the present embodiment, a “plurality of unit pixels” refers to a plurality of the “unit pixel 101 that is a unit pixel”, and a “plurality of pixels” refers to a plurality of pixels with one “pixel” of two, left and right, pixels included in each of the unit pixels 101 as a unit, and each of the “high-sensitivity pixel group” and the “normal sensitivity pixel group” refers to an aggregate with the one “pixel” as a unit.

In the present embodiment, color filters having different optical characteristics for respective “pixels”, what is an on-chip color filter array, are disposed on the respective “pixels” included in the unit pixels 101. In the present embodiment, for the on-chip color filters, filters of complementary colors and primary colors are employed.

Also, the “plurality of pixels” in the light receiving section 22 can be classified into a high-sensitivity pixel group 22a (first pixel group) and a normal sensitivity pixel group 22b (second pixel group) having respective sensitivities that are different from each other according to the characteristics of the on-chip color filters.

Note that here, “high-sensitivity pixel” means a pixel expected to have a relatively high sensitivity in view of a characteristic of illuminating light from a light source and a characteristic of an on-chip color filter disposed on the relevant pixel and a pixel other than the high-sensitivity pixel is defined as “normal pixel” in the present embodiment.

As described above, the image pickup device 21 according to the present embodiment employs what is called a 4-transistor CMOS image sensor as a base, and in the present embodiment, an example in which horizontal 2-pixel sharing pixels are arranged is employed.

As illustrated in FIG. 3, in the present embodiment, the light receiving section 22 mainly includes, left and right photo diodes (PDs) (a left photo diode 111 and a right photo diode 112), a charge conversion section 113, which is called a floating diffusion layer (FD) for charge detection, left and right charge transfer transistors (a left charge transfer transistor 114 and a right charge transfer transistor 115), a charge reset transistor 116, an amplification transistor 117 and a row selection switch transistor 118, for each of the plurality of unit pixels 101.

Also, in the light receiving section 22, a vertical transfer line 119 is disposed for a plurality of unit pixels 101 on each column, respective output ends of the amplification transistors 117 on the column being connected to the vertical transfer line 119, and is connected to the later-described column circuit 26.

The left photo diode (PD) 111 and the right photo diode (PD) 112 are photoelectric conversion elements disposed as a left-right pair for each unit pixel 101 and each of the left photo diode (PD) 111 and the right photo diode (PD) 112 is a photoelectric conversion section configured to perform photoelectric conversion of incident light and accumulate predetermined signal charges.

The left charge transfer transistor 114 and the right charge transfer transistor 115 are a left-right pair of transfer gate transistors disposed so as to correspond to the left photo diode 111 and the right photo diode 112, respectively, for each unit pixel 101.

In other words, the left charge transfer transistor 114 and the right charge transfer transistor 115 are connected to respective cathodes of the left photo diode 111 and the right photo diode 112 to transfer signal charges accumulated in the respective photo diodes (PD) to the charge conversion section 113.

Also, signal lines for a left pixel transfer signal ϕTGL and a right pixel transfer signal ϕTGR outputted from the vertical scanning circuit 24, which is a charge transfer pulse from the timing generating circuit 23 are connected to respective gates of the left charge transfer transistor 114 and the right charge transfer transistor 115.

On/off control of each of the left charge transfer transistor 114 and the right charge transfer transistor 115 is performed by the left pixel transfer signal ϕTGL or the right pixel transfer signal ϕTGR, and when either of the transfer transistors is turned on, the turned-on transfer transistor transfers signal charges accumulated in the left photo diode 111 or the right photo diode 112 to the charge conversion section 113.

In the present embodiment, each of the left charge transfer transistor 114 and the right charge transfer transistor 115 is driven by a signal of a logical product of the left pixel transfer signal ϕTGL or the right pixel transfer signal ϕTGR and an address pointer ΦSEL(N) of a shift register 205.

In other words, an output line of an AND circuit 201 in the vertical scanning circuit 24 is connected to the gate of the left charge transfer transistor 114 to receive an input of a control signal of a logical product of the address pointer ΦSEL(N) that sequentially moves through respective rows in the shift register according to a row selection signal ϕSEL and the left pixel transfer signal ϕTGL.

Likewise, an output line of an AND circuit 202 in the vertical scanning circuit 24 is connected to the gate of the right charge transfer transistor 115, and a control signal of a logical product of the address pointer ΦSEL(N) that sequentially moves through the respective rows of the row selection signal ϕSEL in the shift register and the right pixel transfer signal ϕTGR is inputted to the gate of the right charge transfer transistor 115.

The charge conversion section (FD) 113 is connected to the left charge transfer transistor 114 and the right charge transfer transistor 115, which are charge transfer sections, so that the signal charges accumulated in the left photo diode (PD) 111 or the right photo diode (PD) 112 are transferred to the charge conversion section (FD) 113.

Also, when the left charge transfer transistor 114 or the right charge transfer transistor 115 is turned on, signal charges in the left photo diode 111 or the right photo diode 112 are transferred to the charge conversion section 113 and the charge conversion section 113 converts the signal charges to a voltage.

The charge reset transistor 116 is a reset section configured to perform a reset operation for resetting the charge conversion section (FD) 113, and one end side of the charge reset transistor 116 is connected to a power supply voltage VDD, and the other end side of the charge reset transistor 116 is connected to the charge conversion section 113. Also, a gate of the charge reset transistor 116 is connected to a signal line for a pixel reset signal ϕRST, which is a control signal generated in the timing generating circuit 23 and outputted from the vertical scanning circuit 24.

On/off control of the charge reset transistor 116 is performed according to the pixel reset signal ϕRST, and when the charge reset transistor 116 is turned on, the charge reset transistor 116 releases signal charges accumulated in the charge conversion section 113, the signal charges relating to the left photo diode 111 or the right photo diode 112, to reset the charge conversion section 113 to a predetermined potential.

In the present embodiment, the charge reset transistor 116 is also driven by a signal of a logical product of the pixel reset signal ϕRST and the address pointer ΦSEL(N) of the shift register 205.

In other words, an output line of an AND circuit 203 in the vertical scanning circuit 24 is connected to the charge reset transistor 116, and a control signal of a logical product of the address pointer ΦSEL(N) that sequentially moves through the respective rows in the shift register according to the row selection signal ϕSEL and the pixel reset signal ϕRST is inputted to the charge reset transistor 116.

The amplification transistor 117 is a transistor configured to perform current amplification of the signal charges converted to the voltage by the charge conversion section (FD) 113, and one end side of the amplification transistor 117 is connected to a power supply voltage VDD via the later-described row selection switch transistor 118 and the other end side of the amplification transistor 117 is connected to the vertical transfer line 119, and the amplification transistor 117 configures a source follower jointly with a non-illustrated constant current source connected to the vertical transfer line 119.

Also, the charge conversion section (FD) 113 is connected to a gate of the amplification transistor 117, and the signal charges of the left photo diode 111 or the right photo diode 112, the signal charges being detected and converted to the voltage in the charge conversion section 113 or charges at the time of a reset in the charge conversion section 113, is inputted to the gate of the amplification transistor 117, and the amplification transistor 117 amplifies the voltage or the charges and outputs the resulting voltage or charges toward the vertical transfer line 119.

One end side of the row selection switch transistor 118 is connected to a power supply voltage VDD and the other end side of the row selection switch transistor 118 is connected to the amplification transistor 117. Also, a gate of the row selection switch transistor 118 is connected to a signal line for a pixel read signal ϕX outputted from the timing generating circuit 23.

On/off control of the row selection switch transistor 118 is performed according to the pixel read signal ϕX, and the row selection switch transistor 118 is turned on to select the relevant “row”, and an output signal from the amplification transistor 117 connected to the row selection switch transistor 118 is read and outputted toward the relevant vertical transfer line 119.

Note that in the present embodiment, as with the respective transistors described above, the row selection switch transistor 118 is also driven by a signal of a logical product of the pixel read signal ϕX and the address pointer ΦSEL(N) of the shift register 205.

In other words, an output line of an AND circuit 204 in the vertical scanning circuit 24 is connected to the row selection switch transistor 118, and a control signal of a logical product of the address pointer ΦSEL(N) that sequentially moves through the respective rows in the shift register according to the row selection signal ϕSEL and the pixel read signal ϕX is inputted to the row selection switch transistor 118.

The row selection switch transistor 118 is configured to perform control for reading an output signal of the amplification transistor 117 when the relevant “row” is selected according to the pixel read signal ϕX described above.

The column circuit 26 includes a constant current source IBIAS (not illustrated) provided on the vertical transfer line 119 connected to an output end of the amplification transistor 117. Note that as described above, the amplification transistor 117 and the constant current source IBIAS jointly configure a source follower to read an output signal from the amplification transistor 117 as a voltage signal.

An outline of operation of the image pickup device 21 configured as above will be described. When light resulting from reflection of predetermined illuminating light generated in the light source apparatus 4 (white light or NBI light in the present embodiment) by an object enters the objective optical system 28, the light receiving section 22 receives the object light and the left photo diode 111 and the right photo diode 112 perform predetermined photoelectric conversion and accumulate predetermined signal charges.

Here, the image pickup device 21 selects a row to be read, via the shift register 205 based on the control signal (ϕSEL) from the timing generating circuit 23, and then causes the relevant charge reset transistors 116 to perform a reset operation immediately before transfer in the relevant left charge transfer transistors 114 or the relevant right charge transfer transistors 115, based on the control signal (pixel reset signal ϕRST) from the timing generating circuit 23 to reset the relevant charge detecting sections (FD) 113 to a reset voltage.

Also, in the image pickup device 21, at a predetermined timing, the left charge transfer transistors 114 or the right charge transfer transistors 115 is controlled to be turned on based on a transfer pulse signal (the left pixel transfer signal ϕTGL or the right pixel transfer signal ϕTGR) from the timing generating circuit 23, and signal charges accumulated in the relevant left photo diodes (PD) 111 or the relevant right photo diodes (PD) 112 are transferred to the respective charge detecting sections (FD) 113.

In the image pickup device 21, before and after the transfer pulse signal, the row selection switch transistors 118 are controlled according to the pixel read signal ϕX from the timing generating circuit 23 to cause the reset voltage and a voltage after signal charge transfer in the charge detecting section (FD) 113 to be read as voltage signals by the source follower configured by the amplification transistor 117 and the constant current source IBIAS.

### <Timing Generating Circuit 23, the Vertical Scanning Circuit 24 and Others>

In the present embodiment, the timing generating circuit 23 receives various drive signals (e.g., a clock signal and horizontal/vertical synchronization signals) from the drive control circuit 32 in the video processor 3 and generates various drive signals for driving respective sections (for example, the vertical scanning circuit 24, the horizontal scanning circuit 25, the column circuit 26 and the output circuit 27) inside the image pickup device 21.

In other words, the timing generating circuit 23 generates the row selection signal ϕSEL in addition to the pixel reset signal ϕRST, the pixel read signal ϕX, the left pixel transfer signal ϕTGL and the right pixel transfer signal ϕTGR described above and sends the signals out to the vertical scanning circuit 24.

And, the timing generating circuit 23 sends out a predetermined drive signal also to the horizontal scanning circuit 25 and the output circuit 27 according to a signal from the drive control circuit 32.

The vertical scanning circuit 24 receives various signals described above from the timing generating circuit 23 and outputs the left pixel transfer signal ϕTGL, the right pixel transfer signal ϕTGR, the pixel read signal ϕX and the pixel reset signal ϕRST toward each of unit pixels 101 on a row selected by the shift register 205 according to the row selection signal ϕSEL.

The horizontal scanning circuit 25 sends out the column selection signal ϕCOL sent out from the timing generating circuit 23 under the control of the drive control circuit 32 toward the column circuit 26 on a column-by-column basis. The column circuit 26 receives an input of an output signal from each amplification transistor 117, the output signal being moved to the vertical transfer line 119, in the light receiving section 22, on a column-by-column basis and takes a difference of the output signal of each amplification transistor 117 before and after a pulse signal of the left pixel transfer signal ϕTGL or the right pixel transfer signal ϕTGR described above and sends out the differential signals toward the output circuit 27 on a column-by-column basis in response to a synchronization signal from the horizontal scanning circuit 25.

The output circuit 27 sends out the output signals for the respective columns, which have been outputted from the column circuit 26, to the video processor 3 via a connection cable at a timing based on a control signal from the timing generating circuit 23.

### <All-Pixel Reading and Decimated Reading>

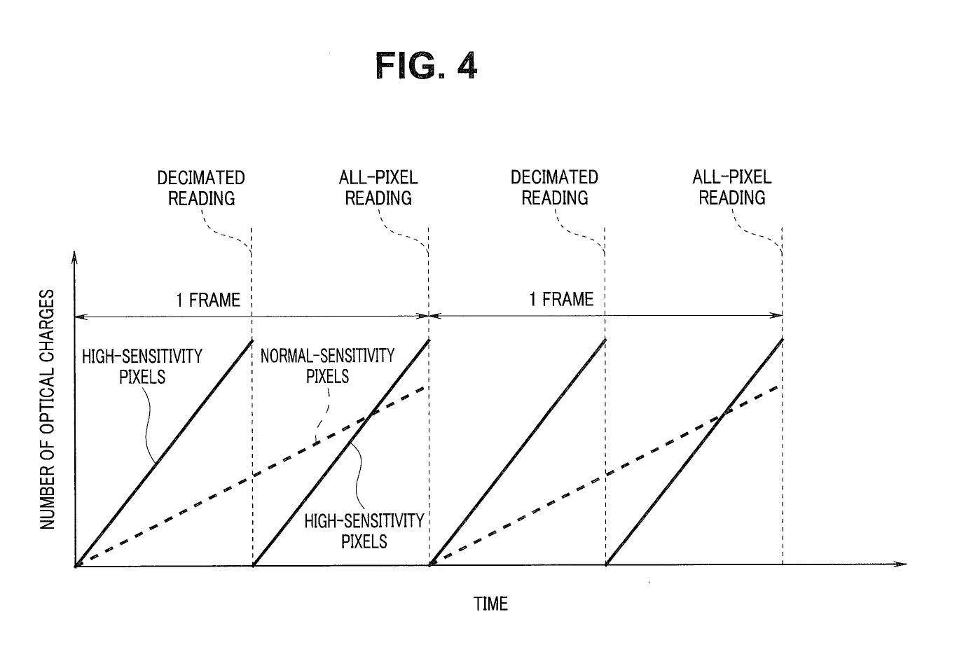

Next, a pixel reading operation in the endoscope system 1 according to the present embodiment will be described with reference to FIGS. 4 to 6.

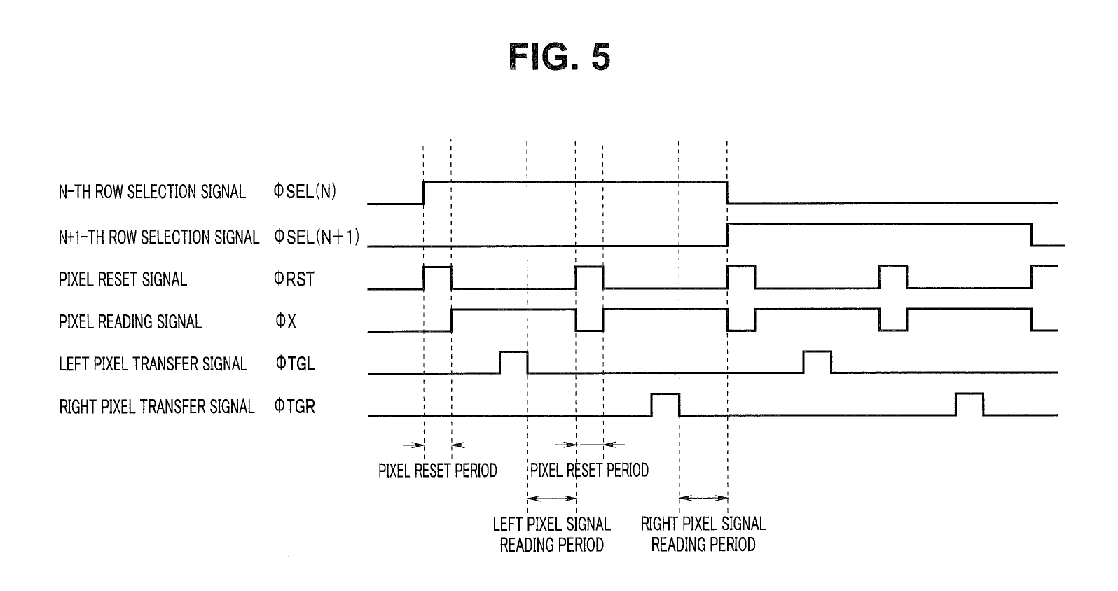

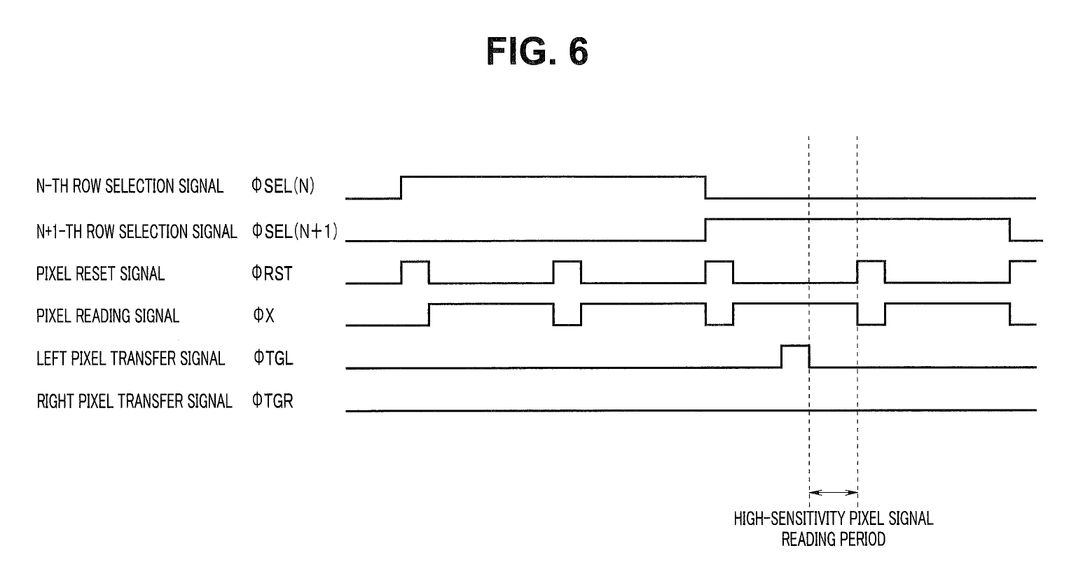

FIG. 4 is a diagram illustrating transition of the respective numbers of optical charges accumulated in photo diodes in high-sensitivity pixels and normal pixels in the image pickup device, the numbers of optical charges being increased with accumulation time, and decimated reading timings and all-pixel reading timings, in the image pickup apparatus according to the first embodiment, FIG. 5 is a timing chart illustrating an example of respective control signals applied to the image pickup device in all-pixel reading, in the image pickup apparatus according to the first embodiment, and FIG. 6 is a timing chart illustrating an example of respective control signals applied to the image pickup device in decimated reading, in the image pickup apparatus according to the first embodiment.

As illustrated in FIG. 4, the image pickup device 21 employed in the present embodiment includes “high-sensitivity pixels” having a high sensitivity relative to normal pixels according to the filter characteristics of the on-chip color filter disposed on the “plurality of pixels” and characteristics of illuminating light applied from the light source apparatus 4 (see the high-sensitivity pixel group 22a (first pixel group) and the normal sensitivity pixel group 22b (second pixel group) illustrated in FIG. 2).

Here, as illustrated in FIG. 4, the high-sensitivity pixels are likely to reach the saturated number of optical charges at a stage before a timing of reading all the pixels in the light receiving section 22 of the image pickup device 21.

The present invention has been made with attention focused on such point and enables effective use of signals generated via light reception in the light receiving section 22, by first performing pixel reading at a stage before saturation of the high-sensitivity pixels (decimated reading) and then performing frame addition of a frame signal resulting from the decimated reading and a frame signal resulting from all-pixel reading (as described later, the frame addition is performed in the frame addition circuit 35 in the video processor 3).

### <All-Pixel Reading Step>

First, an “all-pixel reading step” in the present embodiment will be described with reference to the timing chart illustrated in FIG. 5.

In the present embodiment, under the control of the control section 31 and the drive control circuit 32 of the video processor 3, based on control signals outputted from the timing generating circuit 23 and the vertical scanning circuit 24, outputs of all the pixels in the light receiving section 22 are read at a timing for acquiring one picture (referred to as “1 frame” in FIG. 4) as with a normal pixel reading timing (see “all-pixel reading” in FIG. 4).

In “all-pixel reading”, as illustrated in FIG. 5, first, under the control of the control section 31 and the drive control circuit 32 in the video processor 3, in the vertical scanning circuit 24 that has received a drive signal from the timing generating circuit 23, an N-th row selection signal ϕSEL(N) is controlled to be “H” and an N-th row is thus selected, and the pixel reset signal ϕRST is controlled to be “H” and the output signal from the AND circuit 203 is thus controlled to be “H”.

Consequently, the charge reset transistors 116 on the N-th row in the light receiving section 22 are turned on, and the relevant charge conversion sections (FDs) 113 are reset to the reset voltage.

After the end of the pixel reset period in the charge reset transistors 116, the pixel read signal ϕX from the timing generating circuit 23 is controlled to be “H”, and thus, the signal from the AND circuit 204 is turned to be “H” and the respective row selection switch transistors 118 on the N-th row are turned on.

Here, as a result of the relevant row selection switch transistors 118 being turned on before supply of a pulse of the left pixel transfer signal ϕTGL from the timing generating circuit 23, output signals from the relevant amplification transistors 117 are fed out toward the column circuit 26 via the respective vertical transfer lines 119 and temporarily held in the column circuit 26.

Next, in the timing generating circuit 23, the left pixel transfer signal ϕTGL for the N-th row is controlled to be “H”, and thus, the output signal from the AND circuit 201 in the vertical scanning circuit 24 is turned to be “H”, the gates of the left charge transfer transistors 114 of the relevant horizontal 2-pixel sharing pixels are turned on, and signal charges accumulated in the relevant left photo diodes (PDs) 111 are transferred to the respective charge conversion sections (FDs) 113. Each of the charge conversion sections (FDs) 113 detects the signal charges from the relevant left photo diode (PD) 111 and converts the signal charges to a voltage.

Here, the (accumulated) charges converted to the voltage in the charge conversion sections (FDs) 113 are subjected to current amplification in the respective amplification transistors 117, and since the row selection switch transistors 118 are in an on-state here, the charges based on the left photo diodes (PDs) 111, the charges being amplified in the amplification transistors 117, are fed out to the respective vertical transfer lines 119 and inputted to the column circuit 26.

The column circuit 26 takes a difference of the output signal of each amplification transistor 117 before and after a pulse of the left pixel transfer signal ϕTGL described above, and as described above, feeds signals of the differences out toward the output circuit 27 on a column-by-column basis in response to the synchronization signal from the horizontal scanning circuit 25.

Referring back to FIG. 5, after reading of the left pixel signals as described above, again, in the timing generating circuit 23, the pixel reset signal ϕRST is controlled to be “H”, and thus the output signal from the AND circuit 203 is turned to be “H”, the charge reset transistors 116 on the N-th row are turned on and the relevant charge conversion sections (FDs) 113 are reset to the reset voltage.

Then, after the end of the pixel reset period in the charge reset transistors 116, next, before supply of a pulse of the right pixel transfer signal ϕTGR, in the timing generating circuit 23, the pixel read signal ϕX is controlled to be “H” and the respective row selection switch transistors 118 on the N-th row are turned on again.

Here, as with the above, as a result of the row selection switch transistors 118 being turned on before supply of a pulse of the right pixel transfer signal d? TGR, the output signals from the amplification transistors 117 are fed out toward the column circuit 26 via the respective vertical transfer lines 119 and temporarily held in the column circuit 26.

Subsequently, in the timing generating circuit 23, the right pixel transfer signal ϕTGR for the N-th row is controlled to be “H”, and thus, the output signal from the AND circuit 202 in the vertical scanning circuit 24 is turned to be “H”, the gates of the right charge transfer transistors 115 of the relevant horizontal 2-pixel sharing pixels are turned on and signal charges accumulated in the relevant right photo diodes 112 are transferred to the respective charge conversion sections (FDs) 113. Here, each of the charge conversion sections (FDs) 113 detects the signal charges in the relevant right photo diode (PD) 112 and converts the signal charges to a voltage.

Also, as in the above, since the row selection switch transistor 118 is in an on-state, charges based on the right photo diodes (PDs) 112 that have been amplified by the amplification transistors 117 are fed out to the respective vertical transfer lines 119 and inputted to the column circuit 26.

As in the above-described left pixel signal reading period, the column circuit 26 takes a difference of the output signal of each amplification transistor 117 before and after a pulse of the right pixel transfer signal ϕTGR, and as described above, feeds signals of the differences out toward the output circuit 27 on a column-by-column basis in response to the synchronization signal from the horizontal scanning circuit 25.

Next, in response to a control signal from the timing generating circuit 23, in the vertical scanning circuit 24, an N+1-th row selection signal ϕSEL(N+1) is controlled to be “H” instead of the N-th row selection signal ϕSEL(N) to select the N+1-th row. Subsequently, as with the above-described N-th row selection signal ϕSEL(N), on/off control of the left charge transfer transistors 114 and the right charge transfer transistors 115 on the N+1-th row are performed according to the left pixel transfer signal ϕTGL or the right pixel transfer signal ϕTGR, and thus, left and right pixel signals for the left photo diodes (PDs) 111 and the right photo diodes (PDs) 112 of the relevant horizontal 2-pixel sharing pixels are read.

In the “all-pixel reading step” in the present embodiment, the above-described reading of the left and right pixels (horizontal 2-pixel sharing pixels) on the N-th row and the N+1-th row is performed for all of the pixels, and outputs of the reading of all of the pixels are temporarily stored in a frame memory (not illustrated).

### <Decimated Reading Step>

Next, a “decimated reading step” in the present embodiment will be described with reference to the timing chart illustrated in FIG. 6.

In the present embodiment, when one picture (one frame) is to be generated, only the high-sensitivity pixel group 22a is read at a timing that is other than a timing for the “all-pixel reading” described above (“decimated reading”), and pixel signals of the high-sensitivity pixel group 22a read via the “decimated reading step” are temporarily stored in the frame memory (not illustrated).

In the present embodiment, the “all-pixel reading step” and the “decimated reading step” are performed alternately, and furthermore, a frame signal according to decimated reading and a frame signal according to the above-described all-pixel reading are added up to generate one picture (one frame).

In the present embodiment, description will be given taking an example in which left pixels on even-numbered rows in the light receiving section 22 are “high-sensitivity pixels” as an example of the “decimated reading step”. In other words, in the present embodiment, even-numbered left pixels are “high-sensitivity pixels”; however, the present invention is not limited to the example, and the below-described example is applicable also to, for example, an example in which even-numbered right pixels are “high-sensitivity pixels” and an example in which odd-numbered right pixels or left pixels are “high-sensitivity pixels”.

As illustrated in FIG. 6, as in the “all-pixel reading step”, in “decimated reading” in the present embodiment, first, under the control of the control section 31 and the drive control circuit 32 in the video processor 3, in the vertical scanning circuit 24 that has received a control signal from the timing generating circuit 23, where N is an odd number, the N-th row selection signal ϕSEL(N) is controlled to be “H” and thus, the N-th row that is an odd-numbered row is selected, and the pixel reset signal ϕRST is controlled to be “H” and the output signal of the AND circuit 203 is controlled to be “H”.

Consequently, as in the above, the charge reset transistors 116 on the N-th row, which is one of odd-numbered row in the all of the unit pixels 101 in the light receiving section 22, are turned on, and thus, the relevant charge conversion sections (FDs) 113 are reset to the reset voltage.

Subsequently, in the “decimated reading step” in the present embodiment, also, after the end of the pixel reset period in the charge reset transistors 116, in the timing generating circuit 23, the pixel read signal ϕX is controlled to be “H” and the row selection switch transistors 118 on the N-th row, which is an odd-numbered row, are thus turned on.

As described above, when an odd-numbered row is selected, in the “decimated reading”, as in the “all-pixel reading step”, the charge reset transistors 116 and the row selection switch transistors 118 are turned on.

However, in the “decimated reading step”, when an odd-numbered row is selected, under the control of the control section 31 and the drive control circuit 32 in the video processor 3, in the timing generating circuit 23, the left pixel transfer signal ϕTGL and the right pixel transfer signal ϕTGR are controlled so as not to be “H”.

In other words, when an odd-numbered row is selected in the “decimated reading step” in the present embodiment, the output signals of the AND circuit 201 and the AND circuit 202 in the vertical scanning circuit 24 remain “L”, and therefore, the relevant left charge transfer transistors 114 and the relevant right charge transfer transistors 115 are not turned on and accumulation is continued.

Here, in order to suppress fluctuation of power consumption of the image pickup device 21 arranged at a distal end portion of a cable (not illustrated) and stabilize a power supply voltage supplied via the cable, when an odd-numbered row is selected, the charge conversion sections (FDs) 113 are reset to the reset voltage and the row selection switch transistors 118 are turned on; however, signal output is not caused by these operations and thus may be omitted.

Subsequently, simultaneously with the end of the odd-numbered row-selected period, the shift register 205 receives the row selection signal ϕSEL from the timing generating circuit 23 under the control of the control section 31 and the drive control circuit 32 in the video processor 3 and turns the N+1-th row selection signal ϕSEL(N+1) to be “H” to select the N+1-th row, which is an even-numbered row, instead of the N-th row selection signal ϕSEL(N).

Furthermore, in the timing generating circuit 23, the pixel reset signal ϕRST is controlled to be “H” and the output signal from the AND circuit 203 is controlled to be “H”.

Consequently, the charge reset transistors 116 on the N+1-th row, which is one of the even-numbered row in all of the unit pixels 101 in the light receiving section 22, are turned on and the charge conversion sections (FDs) 113 are thus reset to the reset voltage.

Subsequently, as in the above, after the end of the pixel reset period in the charge reset transistors 116, the pixel read signal 4X from the timing generating circuit 23 is controlled to be “H”, and thus, the signal from the AND circuit 204 is turned to be “H” and the row selection switch transistors 118 on the N+1-th row, which is an even-numbered row, are turned on.

Then, as in the above, as a result of the row selection switch transistors 118 being turned on before supply of a pulse of the left pixel transfer signal ϕTGL from the timing generating circuit 23, output signals from the relevant amplification transistors 117 are fed out toward the column circuit 26 via the respective vertical transfer lines 119 and temporarily held in the column circuit 26.

Next, in the timing generating circuit 23, the left pixel transfer signal ϕTGL for the N+1-th row is controlled to be “H”, and thus, the output signal from the AND circuit 201 in the vertical scanning circuit 24 is turned to be “H”, and the gates of the left charge transfer transistors 114 on the N+1-th row, which is an “even-numbered row”, in the horizontal 2-pixel sharing pixels are turned on, and in the “decimated reading step”, signal charges accumulated in the left photo diodes (PDs) 111 on the “even-numbered row” are transferred to the respective charge conversion sections (FDs) 113. Each of the charge conversion sections (FDs) 113 detects the signal charges from the left photo diode (PDs)111 and converts the signal charges to a voltage.

Here, the charges converted to the voltage in the charge conversion sections (FDs) 113 are subjected to current amplification in the respective amplification transistors 117, and since the row selection switch transistors 118 are in an on-state, as in the above, the charges based on the left photo diodes (PDs) 111, the charges being amplified in the amplification transistors 117, are fed out to the respective vertical transfer lines 119 and inputted to the column circuit 26.

The column circuit 26 takes a difference of the output signal of each amplification transistor 117 before and after a pulse of the left pixel transfer signal ϕTGL described above, and as described above, feeds signals of the differences out toward the output circuit 27 on a column-by-column basis in response to the synchronization signal from the horizontal scanning circuit 25.

Referring back to FIG. 6, after reading of the “even-numbered left pixel signals” as described above, in the “decimated reading step”, again, in the timing generating circuit 23, the pixel reset signal ϕRST is controlled to be “H”, and thus the output signal from the AND circuit 203 is turned to be “H”, the charge reset transistors 116 on an even-numbered row are turned on and the relevant charge conversion sections (FDs) 113 are reset to the reset voltage.

However, in the “decimated reading step”, after the end of the pixel reset period in the charge reset transistors 116, the timing generating circuit 23 does not turn the right pixel transfer signal ϕTGR to be “H” under the control of the control section 31 and the drive control circuit 32 in the video processor 3, that is, the output signal from the AND circuit 202 in the vertical scanning circuit 24 remains “L”, the gates of the right charge transfer transistors 115 in the horizontal 2-pixel sharing pixels are not turned on and accumulation is thus continued.

As described above, in the “decimated reading step” in the present embodiment, under the control of the control section 31 and the drive control circuit 32 in the video processor 3, the timing generating circuit 23 controls only the left pixel transfer signal ϕTGL to be “H” to turn only the left charge transfer transistors 114 on and read only pixel signals of left photo diodes (PDs) 111 in horizontal 2-pixel sharing pixels on each of even-numbered rows.

In other words, in the “decimated reading step” in the present embodiment, subsequently, only pixel signals of left photo diodes (PDs) 111 in horizontal 2-pixel sharing pixels on each of even-numbered rows (as described above, in the present embodiment, “even-numbered left-side pixels” are set as “high-sensitivity pixels”) are fed out from the amplification transistor 117 toward the column circuit 26 via the vertical transfer lines 119.

### <Frame Addition>

In the present embodiment, in the above-described “decimated reading step”, the timing generating circuit 23, the vertical scanning circuit 24, etc., are controlled by the control section 31 and the drive control circuit 32 in the video processor 3 to read left-side pixels in horizontal 2-pixel sharing pixels on even-numbered rows, which correspond to the “high-sensitivity pixel group (first pixel group)” in the light receiving section 22, at a predetermined timing (first reading timing), and outputs resulting from reading of the “even-numbered-row left-side pixels” are temporarily stored as “first frame signals” in the frame memory (not illustrated).

On the other hand, in the present embodiment, in the above-described “all-pixel reading step”, the timing generating circuit 23, the vertical scanning circuit 24, etc., are controlled by the control section 31 and the drive control circuit 32 in the video processor 3 to read all of the pixels including the “high-sensitivity pixel group (first pixel group)” in the light receiving section 22 and the “normal sensitivity pixel group 22b (second pixel group)” in the light receiving section 22 (that is, all of the pixels including left and right pixels (horizontal 2-pixel sharing pixels) on odd-numbered rows and even-numbered rows) at a predetermined timing (second reading timing), and a second frame signal resulting from reading of “all of the pixels” and the first frame signal stored in the frame memory are added up in the frame addition circuit 35 in the video processor 3, and then, in the image processing section 33 of the video processor 3, the resulting frame signal is subjected to image processing for the display apparatus 5 and outputted to the display apparatus 5.

The frame memory may be provided in, for example, the connector 10 in the endoscope 2, may be provided in another part (for example, the vicinity of the endoscope operation portion 8 or the image pickup device 21) in the endoscope 2 or may be provided in, e.g., the image processing section 33 in the video processor 3.

Here, the control section 31, the drive control circuit 32, the timing generating circuit 23 and the vertical scanning circuit 24 described above function as a reading timing control circuit configured to control the second reading timing.

Also, in the present embodiment, the timing generating circuit 23, the vertical scanning circuit 24, etc., are controlled by the control section 31 and the drive control circuit 32 in the video processor 3 to alternately perform the “all-pixel reading step” and “decimated reading step” described above (see FIG. 4). In other words, the control is performed so as to alternately output the first frame signal for the first frame read at the first reading timing and the second frame signal for the second frame read at the second reading timing.

Here, the control section 31, the drive control circuit 32, the timing generating circuit 23 and the vertical scanning circuit 24 described above function as an output control circuit configured to perform control to alternately output the first frame signal and the second frame signal.

Furthermore, in the present embodiment, in the frame addition circuit 35 in the video processor 3, the first frame signal and the second frame signal are subjected to addition processing to generate an image signal for one picture (one frame). More specifically, the first frame signal for the “high-sensitivity pixel group: even-numbered-row left-side pixels” read in the “decimated reading step”, the first frame signal being stored in the frame memory, and the second frame signal for “all of the pixels” read in the “all-pixel reading step” are subjected to addition processing.

As described above, the first embodiment enables provision of an image pickup apparatus (endoscope) including a solid-state image pickup device that includes a color filter array of color filters, the solid-state image pickup device including high-sensitivity pixels as a part, the high-sensitivity pixels each having a sensitivity enhanced by enhancement in transmittance of the relevant color filter, enabling effective use of the high-sensitivity pixel signals and thus always enabling pickup of an image without waste of signal charges.

In the first embodiment, as described above, in the “decimated reading step”, the even-numbered-row left pixels in the light receiving section 22 are “high-sensitivity pixels”; however, the present invention is not limited to the example, and for example, even-numbered-row right pixels, odd-numbered-row right pixels or odd-numbered-row left pixels may be “high-sensitivity pixels”.

Furthermore, high-sensitivity pixels may appropriately be changed or selected. Furthermore, high-sensitivity pixels may dynamically be changed (see the below second embodiment).

### Second Embodiment

Next, a second embodiment of the present invention will be described.

An endoscope system including an image pickup apparatus (endoscope) according to the second embodiment is similar in basic configuration to the endoscope system according to the first embodiment and is different from the endoscope system according to the first embodiment in that in a “decimated reading step”, dynamic change of pixels to be subjected to decimated reading is possible.

Therefore, here, only differences from the first embodiment will be described and description of parts that are in common with the first embodiment will be omitted.

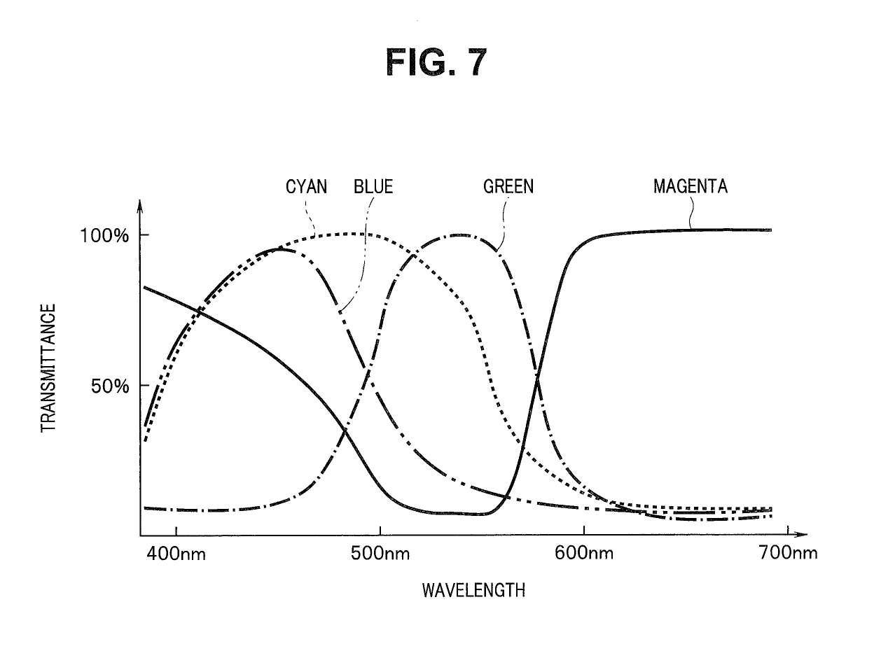

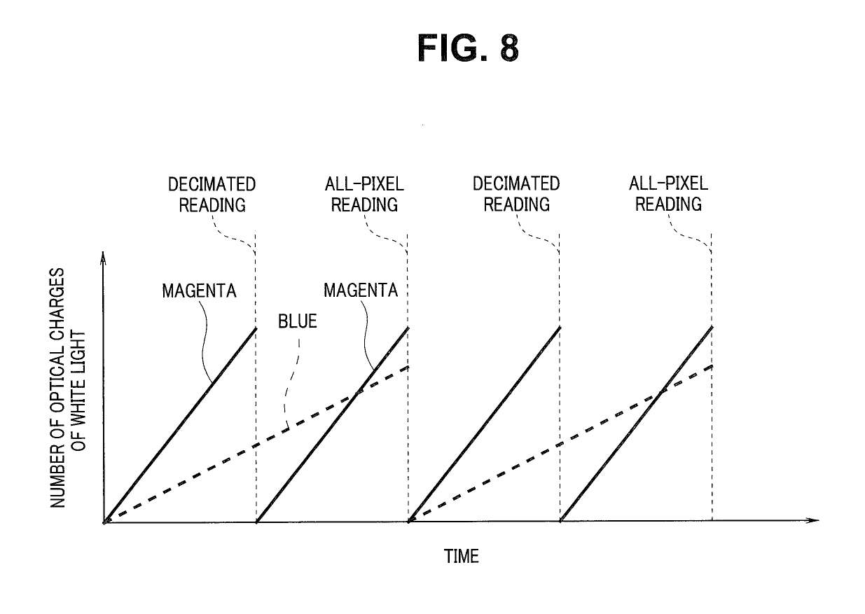

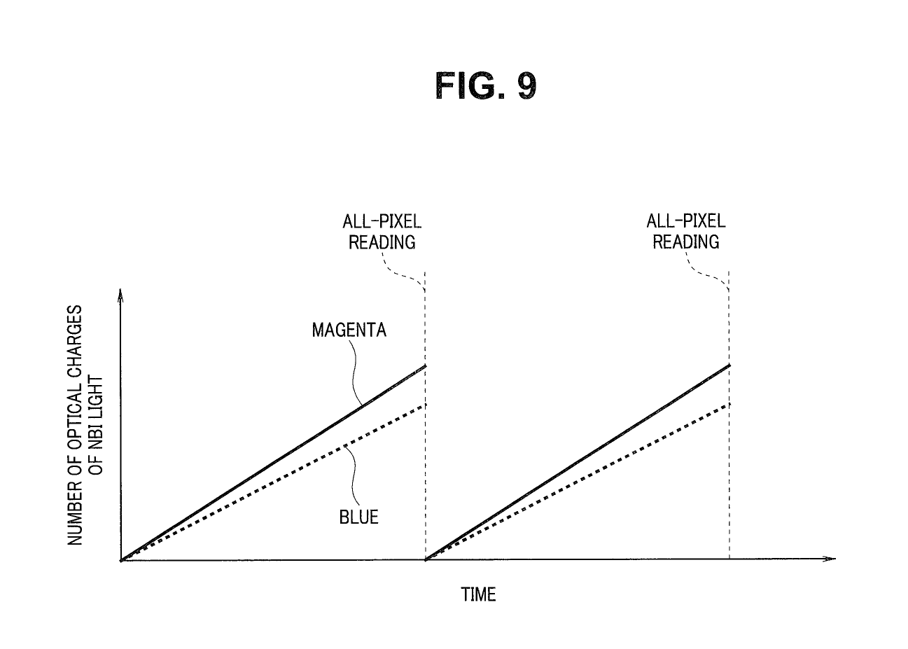

FIG. 7 is a diagram illustrating transmittances of an on-chip color filter where an image pickup device employs primary color filters of respective colors of blue and green in addition to complementary color filters of cyan and magenta in the image pickup apparatus according to the second embodiment of the present invention. FIG. 8 is a diagram illustrating decimated reading timings and all-pixel reading timings when a light source selects white light where the image pickup device includes both complementary color filters and primary color filters disposed in a light receiving section in the image pickup apparatus according to the second embodiment, and FIG. 9 is a diagram illustrating pixel reading timings when the light source selects NBI light where the image pickup device includes both complementary color filters and primary color filters disposed in the light receiving section in the image pickup apparatus according to the second embodiment.

In the second embodiment, as an on-chip color filter, an on-chip color filter formed by combination of primary color filters and complementary color filters is employed for enhancement in sensitivity.

In the second embodiment, as described above, an on-chip color filter formed by combination of primary color filters and complementary color filters is employed, and thus a phenomenon in which respective pixels in the light receiving section 22 have different sensitivities depending on the type of illuminating light from a light source apparatus 4 occurs.

In other words, with reference to the “transmittances of the respective filter colors” in FIG. 7, it can be seen that where illuminating light generated in the light source apparatus 4 is “white light”, which is normal observation light, in the light receiving section 22, cyan pixels (pixels with a “cyan” filter disposed as an on-chip color filter) generally respond to “green light” and “blue light” and magenta pixels (pixels with a “magenta” filter disposed as an on-chip color filter) generally respond to “blue light” and “red light”.

As described above, in the present embodiment, pixels corresponding to complementary color filters each have a sensitivity that is approximately twice a sensitivity of blue pixels (pixels with a “blue” filter disposed as an on-chip color filter) or green pixels (pixels with a “green” filter disposed as an on-chip color filter).

In other words, in the present embodiment, where illuminating light from the light source apparatus 4 is “white light”, in the “decimated reading step”, decimated reading processing is performed with the “cyan pixels” and the “magenta pixels” as “high-sensitivity pixels” (see FIG. 8).

On the other hand, where illuminating light generated in the light source apparatus 4 is NBI light, which is narrow-band observation light (it is assumed that the light source apparatus 4 in the present embodiment provides blue narrow-band light and green narrow-band light), the magenta pixels with the complementary color filters in this case each have a sensitivity that is substantially the same as the sensitivity of the blue pixels with primary color filters because no “red light” is provided as illuminating light.

In other words, in the present embodiment, where illuminating light from the light source apparatus 4 is “NBI light”, in the “decimated reading step”, decimated reading processing is performed with only the “cyan pixels” as “high-sensitivity pixels” and “magenta pixels” are treated as “normal pixels” (see FIG. 9).

More specifically, in the second embodiment, a control section 31 in a video processor 3 performs switching of illuminating light from the light source apparatus 4, that is, switching of the light type between white light and NBI light via a light source selection control signal.

Also, in order to perform dynamic change of pixels that are subjected to decimated reading together with the change of illuminating light, the control section 31 feeds the light source selection control signal from a drive control circuit 32 to a timing generating circuit 23 in the endoscope 2.

Then, in the present embodiment, when the timing generating circuit 23 has received the light source selection control signal from the drive control circuit 32, based on the signal, the timing generating circuit 23 performs processing for changing “pixels” to be read as “high-sensitivity pixels” in such a manner as described above in the “decimated reading step”.

In other words, if the timing generating circuit 23 recognizes that illuminating light from the light source apparatus 4 is “white light”, based on the light source selection control signal from the drive control circuit 32, the timing generating circuit 23 performs decimated reading processing with the “cyan pixels” and the “magenta pixels” as “high-sensitivity pixels” in the “decimated reading step”, and on the other hand, if the timing generating circuit 23 recognizes that illuminating light from the light source apparatus 4 is “NBI light”, the timing generating circuit 23 performs decimated reading processing with only the “cyan pixels” as “high-sensitivity pixels” in the “decimated reading step” and treats the “magenta pixels” as “normal pixels” and performs reading processing for the “magenta pixels” only at timings for all-pixel reading.

As described above, as in the first embodiment, the second embodiment enables provision of an image pickup apparatus (endoscope) including a solid-state image pickup device that includes a color filter array, the solid-state image pickup device including high-sensitivity pixels as a part, enabling effective use of high-sensitivity pixel signals and thus always enabling pickup of an image without waste of signal charges because pixels having a risk of being saturated can selectively be read depending on the light source.

### <Modification>

Each of the endoscope systems 1 according to the respective embodiments described above includes the light source apparatus 4 configured to generate NBI light, which is narrow-band observation light, and white light, which is normal observation light. In order to obtain a color image with normal white light, an on-chip color filter is arranged in the image pickup device 21. Where the on-chip color filter is of a type that absorbs narrow-band observation light, problems that, for example, an image during observation using narrow-band observation light becomes dark or that desired resolution cannot be obtained occur.

On the other hand, in recent years, pixels have become increasingly finer, and if a resolution of a lens is enhanced so as to respond to a pixel pitch of such finer pixels, an f-number (aperture value) needs to be decreased, resulting in shallowing of a depth of field. In order to obtain a desired depth of field, a problem that the resolution is sacrificed, for example, occurs.

Therefore, the present modification will be described in terms of an image pickup apparatus that enables overcoming a trade-off between a depth of field and resolution, which occurs as a result of pixels becoming finer and thus enables observation with both narrow-band observation light and normal observation light.

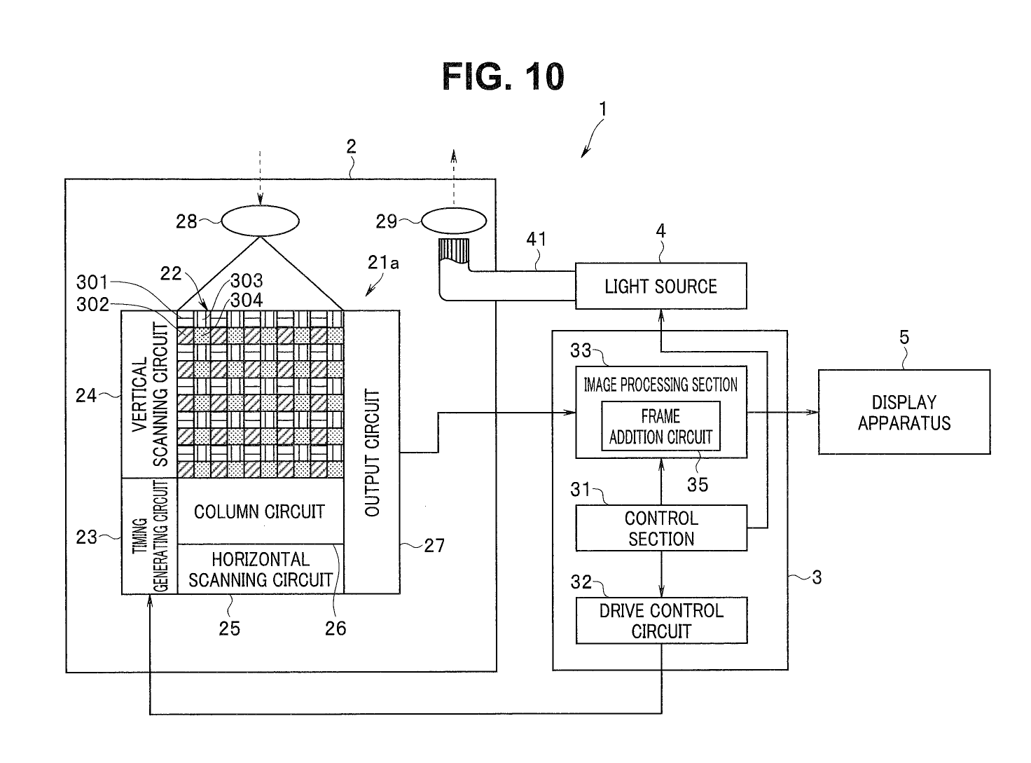

FIG. 10 is a block diagram illustrating an electric configuration of an endoscope system including an image pickup apparatus according to the modification. Note that in FIG. 10, components that are similar to components in FIG. 2 are provided with reference numerals that are the same as the reference numerals of the components and description of such components will be omitted.

As illustrated in FIG. 10, an on-chip color filter included in an image pickup device 21a includes G filters 301 configured to mainly transmit a wavelength band of green light, R filters 302 configured to mainly transmit a wavelength band of red light, B filters 303 configured to mainly transmit a wavelength band of blue light and Cy filters 304 configured to mainly transmit wavelength bands of green light and blue light.

The G filters 301, the R filters 302, the B filters 303 and the Cy filters 304 are arranged on respective pixels in a light receiving section 22. More specifically, as illustrated in FIG. 10, a G filter 301 is arranged on a pixel in the upper left of the light receiving section 22, a R filter 302 is arranged on a pixel below the pixel with the G filter 301 arranged, a B filter 303 is arranged on a pixel to the right of the pixel with the G filter 301 arranged, and a Cy filter 304 is arranged on a pixel to the right of the pixel with the R filter 302 arranged.

The G filters 301 are arranged with a pitch of two pixels in vertical and horizontal directions. Likewise, the respective R filters 302 are arranged with a pitch of two pixels in the vertical and horizontal directions. Likewise, the respective B filters 303 are arranged with a pitch of two pixels in the vertical and horizontal directions. Likewise, the respective Cy filter 304 are arranged with a pitch of two pixels in the vertical and horizontal directions. As a result, in the on-chip color filter included in the image pickup device 21a, numbers of G filter 301, R filter 302, B filter 303 and Cy filter 304, the numbers being equal to one another, are arranged.

At the time of observation with narrow-band observation light using blue light and green light, an image processing section 33 in a video processor 3 performs color separation for the pixels corresponding to the G filters 301, the pixels corresponding to the B filters 303 and the pixels corresponding to the Cy filters 304 to generate an image signal. On the other hand, at the time of observation with normal observation light using white light, the image processing section 33 in the video processor 3 performs color separation for the pixels corresponding to the G filters 301, the pixels corresponding to the R filters 302, the pixels corresponding to the B filters 303 and the pixels corresponding to the Cy filters 304 to generate an image signal.

A general image pickup device includes an on-chip color filter with G filters, R filters and B filters arranged in a pattern of a Bayer array. In a Bayer array, G filters that generate a luminance signal are arranged in a checkered pattern, and thus, in order to effectively utilize the resolution of the image pickup device, a lens of an objective optical system is required to have a lens resolution that is √2 times a pixel pitch.

On the other hand, the image pickup device 21a according to the present modification includes an on-chip color filter in which the Cy filters 304 that generate a luminance signal are arrayed in a grid pattern in addition to the G filter 301, the R filters 302 and the B filters 303. Therefore, in order to effectively utilize the resolution of the image pickup device 21a, a lens of an objective optical system 28 only needs to have a lens resolution that is twice the pixel pitch.

Furthermore, the Cy filters 304 have sensitivity to both blue light and green light, which are mainly used for narrow-band observation light, being able to suppress resolution decrease at the time of observation using narrow-band observation light.

As a result, an image pickup apparatus (endoscope 2) including the image pickup device 21a according to the present modification can overcome a trade-off between a depth of field and resolution, the trade-off occurring as a result of pixels becoming finer, and thus enables observation with both narrow-band observation light and normal observation light.

Note that the arrangement of the G filters 301, the R filters 302, the B filters 303 and the Cy filters 304 of the on-chip color filter included in the image pickup device 21a are not limited to the arrangement in FIG. 10.

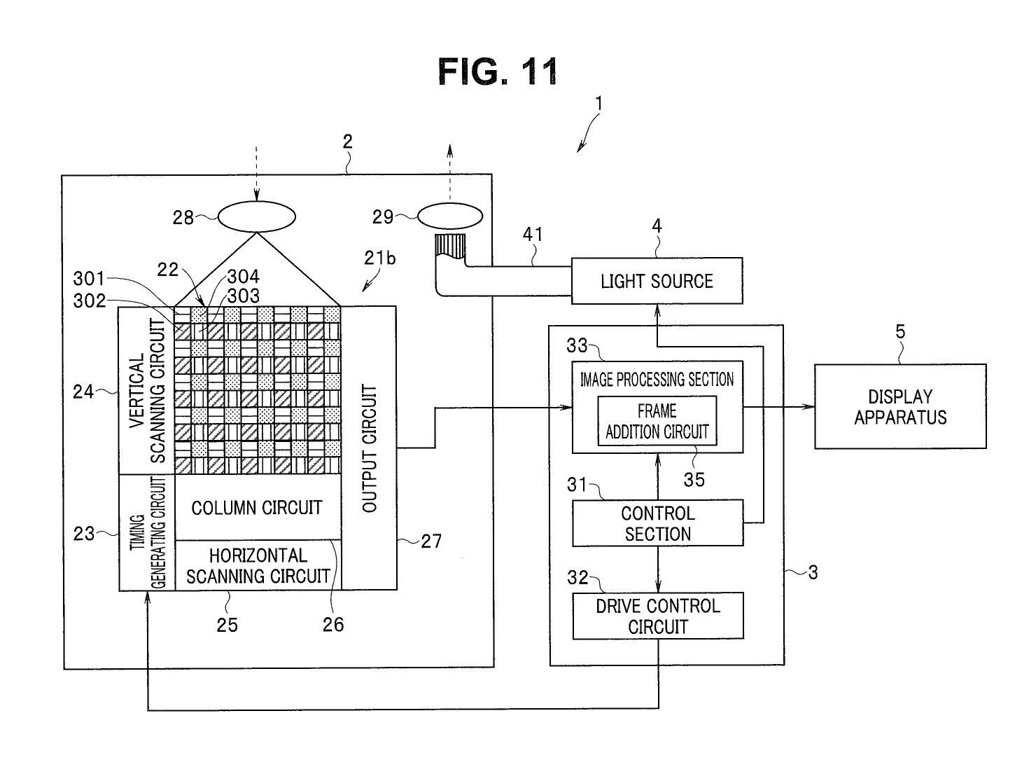

Each of FIGS. 11, 12 and 13 is a block diagram illustrating an electric configuration of an endoscope system including another image pickup apparatus according to the modification. In FIGS. 11 to 13, components that are similar to components in FIG. 10 are provided with reference numerals that are the same as the reference numerals of the components and description of such components will be omitted.

As illustrated in FIG. 11, as with the image pickup device 21a in FIG. 10, an on-chip color filter included in an image pickup device 21b includes G filters 301, R filters 302, B filters 303 and Cy filters 304. The image pickup device 21b illustrated in FIG. 11 includes arrangements of the B filter 303 and the Cy filter 304 changed from the arrangements in the image pickup device 21a in FIG. 10.

More specifically, a G filter 301 is arranged on a pixel on the upper left of a light receiving section 22, a R filter 302 is arranged on a pixel below the pixel with the G filter 301 arranged, a Cy filter 304 arranged on a pixel to the right of the pixel with the G filter 301 arranged, and a B filter 303 is arranged on a pixel to the right of the pixel with the R filter 302 arranged. The G filters 301, the R filters 302, the B filters 303 and the Cy filters 304 are arranged with a pitch of two pixels in vertical and horizontal directions, respectively. The rest of the configuration is similar to the configuration of the image pickup device 21a in FIG. 10.

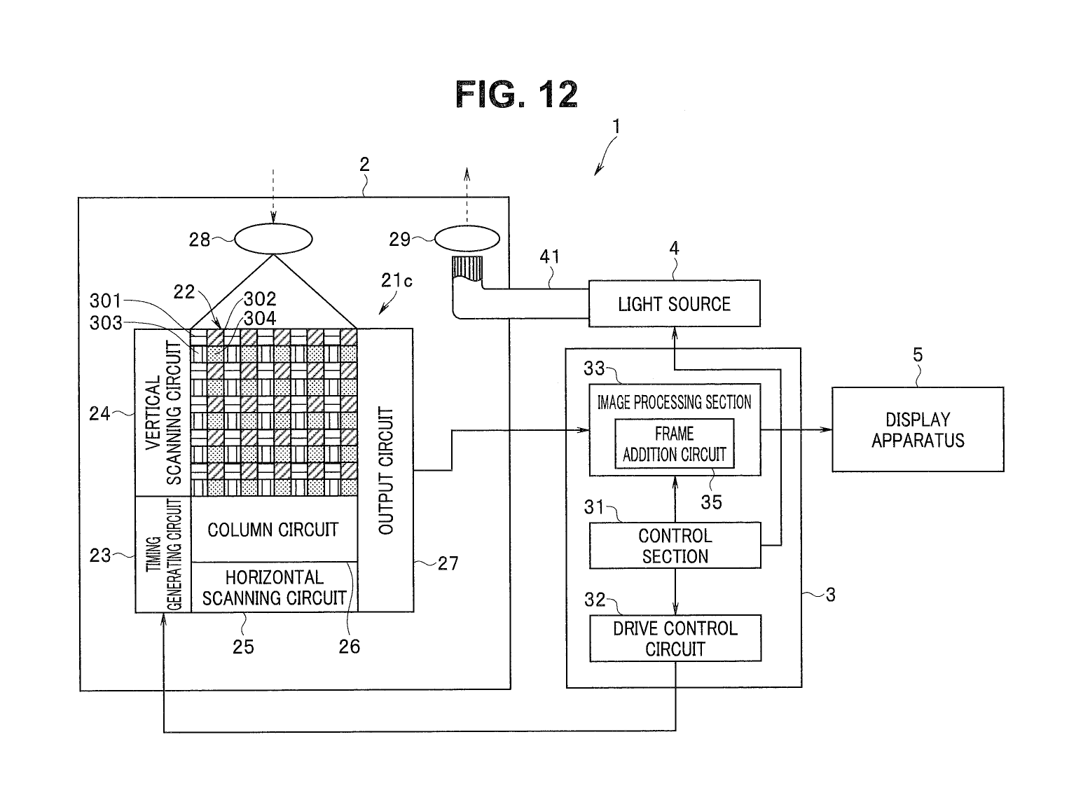

An image pickup device 21c, which is illustrated in FIG. 12, includes arrangements of R filters 302 and B filters 303 changed from the arrangements in the image pickup device 21a in FIG. 10.

More specifically, a G filter 301 is arranged on a pixel in the upper left of a light receiving section 22, a B filter 303 is arranged on a pixel below the pixel with the G filter 301 arranged, a R filter 302 is arranged on a pixel to the right of the pixel with the G filter 301 arranged, and a Cy filter 304 is arranged on a pixel to the right of the pixel with the B filter 303 arranged. The G filters 301, the R filters 302, the B filters 303 and the Cy filters 304 are arranged with a pitch of two pixels in vertical and horizontal directions, respectively. The rest of the configuration is similar to the configuration of the image pickup device 21a in FIG. 10.

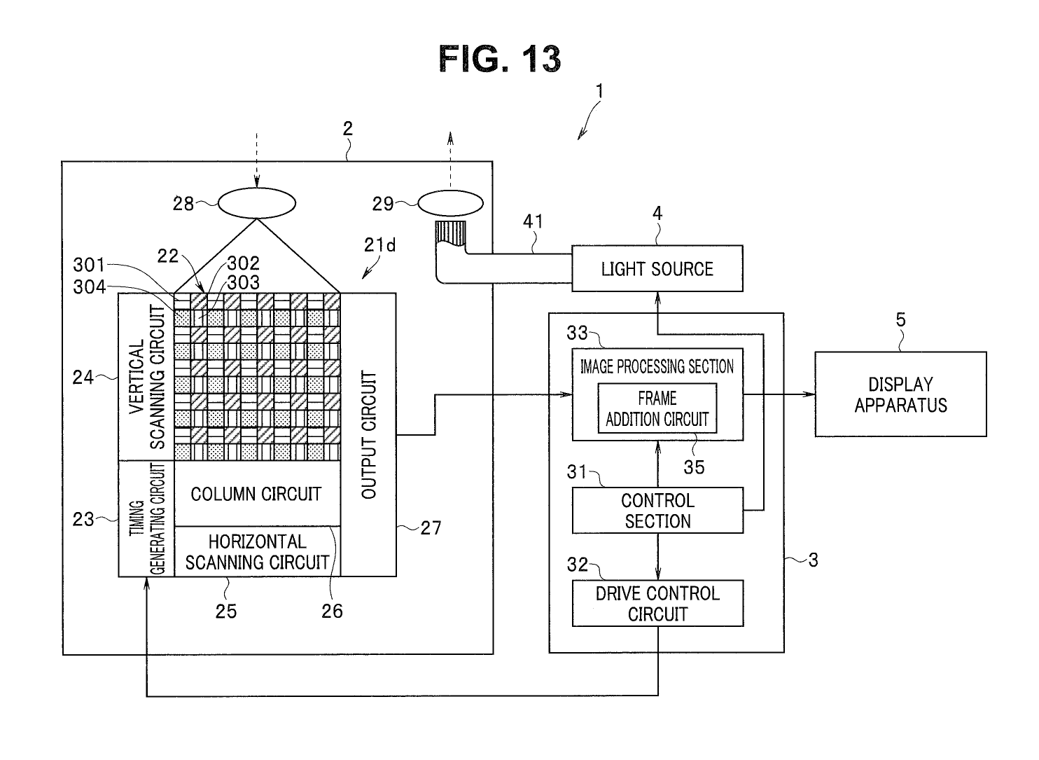

An image pickup device 21d, which is illustrated in FIG. 13, includes arrangements of R filters 302, B filters 303 and Cy filters 304 changed from the arrangements in the image pickup device 21a in FIG. 10.

More specifically, a G filter 301 arranged on the upper left of a light receiving section 22, a Cy filter 304 is arranged on a pixel below the pixel with the G filter 301 arranged, a R filter 302 is arranged on a pixel to the right of the pixel with the G filter 301 arranged, and a B filter 303 is arranged on a pixel to the right of the pixel with the Cy filter 304 arranged. The G filters 301, the R filters 302, the B filters 303 and the Cy filters 304 are arranged with a pitch of two pixels in vertical and horizontal directions, respectively. The rest of the configuration is similar to the configuration of the image pickup device 21a in FIG. 10.

As with the image pickup apparatus including the image pickup device 21a, each of image pickup apparatuses (endoscopes 2) including the image pickup devices 21b, 21c and 21d, respectively, enables overcoming a trade-off between a depth of field and resolution, which occurs as a result of pixels becoming finer, and thus enables observation with both narrow-band observation light and normal observation light.

The present invention is not limited to the above-described embodiments, and various changes, alterations and the like are possible without departing from the spirit of the present invention.

What is claimed is:

1. An image pickup apparatus comprising:
an image pickup device including a color filter array of color filters, in which a first pixel group and a second pixel group including respective sensitivities that are different from each other according to characteristics of the color filters are arranged;
a reading timing control circuit configured to control a first reading timing for a first frame for which a configuration of the first pixel group to be read is appropriately changed according to a control signal synchronized with a light source selection control signal for selecting a type of illuminating light from a light source capable of emitting a plurality of types of illuminating light for illuminating an object, the first frame being forming by reading the first pixel group alone, and a second reading timing for a second frame formed by reading all pixels including the first pixel group and the second pixel group;
an output control circuit configured to, based on the control by the reading timing control circuit, perform control to alternately output a first frame signal for the first frame read at the first reading timing and a second frame signal for the second frame read at the second reading timing; and
a frame addition circuit configured to perform frame addition processing of the first frame signal and the second frame signal controlled and outputted by the output control circuit to output an image signal for one frame.

2. The image pickup apparatus according to claim 1, wherein where the color filter array includes at least a complementary color filter array and the light source is capable of emitting either illuminating light of white light or illuminating light of narrow-band observation light configured by light of one or more single colors as the illuminating light, the reading timing control circuit selects subject pixels for the first pixel group to be read for the first frame in such a manner that the subject pixels can be changed between a case where the illuminating light of white light is selected as the illuminating light and a case where the illuminating light of narrow-band observation light is selected as the illuminating light, according to the predetermined control signal synchronized with the light source selection control signal.

3. The image pickup apparatus according to claim 2, wherein the reading timing control circuit selects cyan pixels and magenta pixels as the subject pixels for the first pixel group to be read for the first frame if the white light illuminating light is selected as the illuminating light, and selects only cyan pixels as the subject pixels for the first pixel group to be read for the first frame if the illuminating light of narrow-band observation light is selected as the illuminating light.

4. The image pickup apparatus according to claim 1, wherein in the image pickup device, horizontal 2-pixel sharing pixels are arranged.

5. The image pickup apparatus according to claim 1, wherein the reading timing control circuit controls a reading timing for the image pickup device according to a wavelength of the illuminating light applied from the light source capable of emitting a plurality of types of illuminating light for illuminating an object, and selects pixels each including sensitivity raised according to the illuminating light from the light source, as the first pixel group.

6. The image pickup apparatus according to claim 1, wherein:
the color filters included in the image pickup device include G filters configured to transmit a wavelength band of green light, R filters configured to transmit a wavelength band of red light, B filters configured to transmit a wavelength band of blue light, and Cy filters configured to transmit wavelength bands of green light and blue light; and
the G filters, the R filters, the B filters and the Cy filters are arranged with a pitch of two pixels in vertical and horizontal directions, respectively.

7. The image pickup apparatus according to claim 6, wherein:
each of the G filters is arranged on a predetermined pixel in a light receiving section of the image pickup device;
each of the R filters is arranged on a pixel below the predetermined pixel with the relevant G filter arranged;
each of the B filters is arranged on a pixel to a right of the predetermined pixel with the relevant G filter arranged; and
each of the Cy filters is arranged on a pixel to a right of the pixel with the relevant R filter arranged.

8. The image pickup apparatus according to claim 6, wherein:
each of the G filters is arranged on a predetermined pixel in a light receiving section of the image pickup device;
each of the R filters is arranged on a pixel below the predetermined pixel with the relevant G filter arranged;
each of the Cy filters is arranged on a pixel to a right of the pixel with the relevant G filter arranged; and
each of the B filters is arranged on a pixel to a right of the predetermined pixel with the relevant R filter arranged.

9. The image pickup apparatus according to claim 6, wherein:
each of the G filters is arranged on a predetermined pixel in a light receiving section of the image pickup device;
each of the B filters is arranged on a pixel below the predetermined pixel with the relevant G filter arranged;
each of the R filters is arranged on a pixel to a right of the predetermined pixel with the relevant G filter arranged; and
each of the Cy filters is arranged on a pixel to a right of the pixel with the relevant B filter arranged.

10. The image pickup apparatus according to claim 6, wherein:
each of the G filters is arranged on a predetermined pixel in a light receiving section of the image pickup device;
each of the Cy filters is arranged on a pixel below the predetermined pixel with the relevant G filter arranged;
each of the R filters is arranged on a pixel to a right of the predetermined pixel with the relevant G filter arranged; and
each of the B filters is arranged on a pixel to a right of the pixel with the relevant Cy filter arranged.

11. An endoscope comprising the image pickup apparatus according to claim 1.

12. An endoscope system comprising the endoscope according to claim 11.
